(19) United States
(12) Patent Application Publication
(10) Pub. No.: US 2013/0102538 A1
(43) Pub. Date: Apr. 25, 2013

(54) METHOD OF TREATING ALZHEIMER'S DISEASE USING PHARMACOLOGICAL CHAPERONES TO INCREASE PRESENILIN FUNCTION AND GAMMA-SECRETASE ACTIVITY

(71) Applicants: Brandon Wustman, San Diego, CA (US); Anthony Stevens, San Diego, CA (US)

(72) Inventors: Brandon Wustman, San Diego, CA (US); Anthony Stevens, San Diego, CA (US)

(73) Assignee: AMICUS THERAPEUTICS, INC., Cranbury, NJ (US)

(21) Appl. No.: 13/696,000

(22) PCT Filed: May 4, 2011
(86) PCT No.: PCT/US11/35182

§ 371 Date: Jan. 10, 2013

Related U.S. Application Data
(60) Provisional application No. 61/331,735, filed on May 5, 2010.

(51) Int. Cl. A61K 31/55; A61K 38/05; A61K 38/08; A61K 31/222; A61K 31/27; A61K 31/5513; A61K 31/196; A61K 31/166

(52) U.S. Cl. CPC A61K 31/55; A61K 31/196; A61K 38/05; A61K 31/166; A61K 31/222; A61K 31/27; A61K 31/5513; A61K 38/08
USPC 514/17.6; 514/212.04; 514/562; 514/17.8; 514/616; 514/539; 514/486; 514/221; 514/18.2

(57) ABSTRACT

The present invention relates to a method for treating an individual having Alzheimer's Disease by using pharmacological chaperones that bind presenelin and thereby increase γ-secretase activity.

### FIELD OF THE INVENTION

The present invention relates to a method for treating an individual having Alzheimer's Disease by using pharmacological chaperones that bind presenelin and thereby increase γ-secretase activity.

### BACKGROUND OF THE INVENTION

Alzheimer's Disease is one of the largest socioeconomic healthcare burdens. Alzheimer's disease is characterized by progressive dementia and histopathologically by the presence of neurofibrillary tangles (NFTs) and neuritic (senile) amyloid plaques. Amyloid plaques mainly consist of a protein fragment called Aβ42 and tangles are predominantly made up of a protein called Tau.

Aβ is a hydrophobic 38- to 43-amino acid peptide, found in all biological fluids, and derived from the progressive enzymatic cleavage of a larger type I membrane protein, the amyloid precursor protein (APP). Gamma-secretase (γ-secretase) is a multiprotein enzymatic complex that is responsible for generating the different Aβ species from truncated APP called c-terminal fragments (CTFs), which is generated by either alpha or beta secretase activity. γ-secretase generated Aβ is only produced from beta-secretase processed APP (FIG. 1). Genetic linkage studies of early onset familial AD patients identified a number of mutations in three genes, APP, PSEN1 and PSEN2. PSEN1 and PSEN2 encode γ-secretase proteins presenilin 1 (PS1) and presenilin 2 (PS2), respectively. APP and the presenilins (components of γ-secretase) are genetically linked to early onset Alzheimer's Disease.

Early Onset Familial Alzheimer's disease (EOFAD) is a characterized by autosomal dominant inheritance of mutations in PSEN1, PSEN2 or APP, resulting in early onset AD (100% penetrance by age 65). Greater than 60% of EOFAD cases are a result of mutations in the PSEN1 gene (Alzheimer's Type 3 or AD3) and the vast majority of those mutations are missense mutations generating a single amino acid change in the PS1 primary structure. It is estimated the number of PS1 FAD cases are between 25,000 and 100,000 affected people in the US.

γ-secretase is an aspartyl protease that cleaves type I membrane proteins within the transmembrane domain. All four protein components that make up the γ-secretase complex, presenilin (presenilin 1 or presenilin 2), aph1, nicastrin and pen2, are required for proteolytic activity. Presenilin 1 and 2 are highly homologous (74%) polytopic membrane proteins consisting of nine transmembrane domains and possesses the active site for γ-secretase proteolytic activity. Presenilin is translated in the ER as a 45 kDa precursor chain where it is joined by two other proteins aph1 and nicastrin to form a trimer. The presenlin/aph1/nicastrin trimer exits the ER and in an early golgi compartment is joined by Pen2, the fourth member of the tetrameric complex. Upon pen2 forming the tetrameric complex, presenilin undergoes cleavage at residues between TM6 and TM7 to form a presenilin heterodimer consisting of a 27 kDa N-terminal fragment and a 18 kDa C-terminal fragment. The cleavage of presenilin, also known as presenilinase activity, is thought to occur through an autocatalytic event by the γ-secretase complex. After proteolytic processing of PS1 the γ-secretase complex is considered to be in its active form. The presenilin precursor in the ER has a very short half-life of ˜1 hour while the mature form of presenilin (in complex with aph1, nicastrin and pen2) has a considerably longer half-life of ˜24 hours (Ratovitski et al. 1997).

Presenilin is a multifunctional protein that's involved in a number of different cellular processes including nuclear signal transduction, protein trafficking, intracellular calcium homeostasis, hippocampal neurogenesis and apoptosis. While most therapies are focused on inhibiting gamma-secreatse activity to reduce Aβ generation, a number of other presenilin 1 functions have been shown to be impaired by mutations in the gene encoding presenilin-1 (PSEN1) (see Table 1).

TABLE 1
Summary of presenilin 1 functions and loss-
of-functions caused by mutations in PSEN1.

| Presenilin 1 function | Effects of presenilin loss-of-function |
|---|---|
| Amyloid precursor protein | Reduced Aβ40 production relative to |
| (APP) processing | Aβ42, reduced neprilysin activity |
| Notch, Delta1 and Jagged2 | Impaired notch signaling may alter |
| processing | hippocampal neurogenesis |
| EphB processing | Altered/impaired angiogenesis, axonal |
| | guidance, and neuronal plasticity |
| ErbB4 processing | NRG1/erbB4 signaling perturbation, |
| | neurodevelopmental deficit, and |
| | glutamatergic hypofunction. |
| N-cadherin processing | Dysregulation of CBP/CREB-dependent |
| | transcription required for function and |
| | plasticity of the nervous system |
| Lipoprotein receptor (LRP1) | Dysregulation of apoE and cholesterol |
| processing | levels in the CNS |
| Leukocyte-common antigen- | Dysregulation of cell adhesion, |
| related (LAR) processing | formation of functional synapses and |
| | neuronal networks |
| Nectin-1α processing | Impaired synapse formation |
| APLP, E-cadherin and CD44 | Impaired nuclear signaling pathways |
| processing | |
| PS1 activation of the PI3K/Akt | Down regulation of PI3/Akt signaling |
| signaling pathway | and PTEN causing impaired cell |
| | proliferation, migration and apoptosis |
| | and increased tau phosphorylation |
| Regulates environmental | Impaired hippocamal neurogenesis |
| enrichment-mediated neural | |
| progenitor cell proliferation and | |
| neurogenesis in adult | |
| hippocampus | |
| Trafficking/sorting of APP | Reduced trafficking of APP to the |
| | plasma membrane, increase Aβ |
| | production and reduced sAPPα |
| | generation |
| Trafficking and turnover of | Impaired hippocampal neurogeneis |
| epidermal growth factor | |
| receptor (EGFR) | |
| Processing of SorLA, Sortilin | Altered or impaired protein trafficking |
| and SorCS1b | and sorting |
| Stabilization of β-catenin | Reduced stabilization of β-catenin |
| | leading to increased vulnerability of |
| | neurons to apoptosis |
| Interact and modulate | Impaired maturation of autophagic |
| trafficking of telencephalin | vesicles |
| Regulate SERCA pump activity | Altered Ca2+ signaling, impaired |
| | calcium homeostasis, reduced calcium |
| | levels in the ER |
| Passive ER Ca2+ leak channels | Reduced ER Ca2+ leak disturbing |
| | calcium homeostasis and calcium |
| | signaling |

Although the γ-secretase complex is best known as an intramembrane protease where over 50 type I membrane proteins are known to be cleaved within the transmembrane domain (gamma site), γ-secretase also cleaves the membrane protein at a site juxtaposed to the cytosol/membrane interface (epsilon site) releasing an intracellular domain (ICD). In many instances the ICD is translocated to the nucleus where it is involved in regulating gene expression. For example, γ-secretase plays a key role in the notch signaling pathway. When membrane protein receptor notch engages with one of its membrane bound ligands (delta-like and jagged) the extracellular domain of notch is cleaved by an alpha secretase (ADAM10/17) and released leaving behind a membrane bound c-terminal fragment (CTF). The notch CTF is processed by γ-secretase releasing notch intracellular domain (NICD) which is translocated into the nucleus where it forms a complex with the DNA binding protein CSL, leading to the transcriptional activation of notch target genes. Indeed, γ-secretase inhibitors used to treat Alzheimer's disease have been plagued by severe GI side effects associated with notch inhibition.

An analogous process takes place with APP where its intracellular cytoplasmic domain, AICD, is released by γ-secretase leading to transcriptional activation of APP target genes. In fact, these signaling pathways mediated by γ-secretase are quite prevalent involving many different membrane receptors including ErbB4, APLP, E-cadherin, N-cadherin, CD44, nectin1, p75NTR, LRP, Delta1, Jagged2 and LAR. Presenilin has also been shown to effect nuclear signaling through a γ-secretase independent mechanism. The wnt/frizzeled signaling pathway leads to beta-catenin stabilization, nuclear translocation, gene activation and cell proliferation. Presenilin has been shown to interact with beta-catenin and suppress its ability for nuclear entry and gene activation.

Presenilin 1 (PS1) has been shown to have a role in protein trafficking in both a γ-secretase dependent and independent manner. Proteins SorLA, Sortilin and SorCS1b, members of the Vps10p family of sorting receptors involved in protein sorting and trafficking of many different proteins, are also substrates of γ-secretase and therefore their functions could be compromised in cells with lower than normal γ-secretase activity. FAD PS1 mutants have been shown to decrease the trafficking of APP to the cell surface resulting in greater beta-processing of APP and increased Aβ levels, as well as decreased levels of the beneficial sAPPalpha. Autophagy is a cellular process involved in organelle degradation/recycling that has been shown to be upregulated during neurodegeneration. It's been hypothesized that inefficient maturation of autophagosomes can lead to accumulation of these structures in neurons leading to AD pathology. Presenilins have been reported to interact and modulate the trafficking of telencephalin, effecting the maturation of autophagic vesicles. A deficiency in PS1 resulted in pre-autophagic vesicles unable to mature through fusion with endosome/lysosomes.

The importance of calcium homeostasis in neuronal viability has lead to multiple studies suggesting that cellular calcium regulation may also play a role in Alzheimer's disease. Presenilins have been shown to be involved in cellular calcium homeostasis where PS1 FAD mutants were reported to increase ER calcium capacitative entry and calcium leak activity.

It is now well accepted that neurogenesis takes place during brain development and in the adult animal. It's believed that neurogenesis in the adult may play a role in learning and memory, two tasks dramatically affected in patients with Alzheimer's disease. Neurogenesis has been shown to increase in mice that are housed in an enriched environment. However, it was found that neurogenesis is not stimulated in an enriched housing environment in mice expressing presenilin FAD mutations, leading researchers to suggest that PS1/γ-secretase loss-of-function might be tied to decreased neurogenesis and Alzheimer's disease.

One of the hallmarks of Alzheimer's disease as well as other neurodegenerative diseases is selective neuronal loss, thought to be driven by the process of apoptosis. Many FAD PS1 mutations have been shown to make neurons more vulnerable to apoptosis. Neurons deficient in PS1 were shown to have increased caspase-3 activity leading to apoptosis. The PS1 deficient neurons could be rescued from caspase-3 activation and apoptosis by expression of wildtype PS1, but not PS1 with FAD mutations.

One of the current pharmaceutical approaches for treating Alzheimer's disease is to inhibit γ-secretase activity using γ-secretase inhibitors and thereby block the production of Aβ. Studies have shown that most mutations in PS1 lead to an increase in the Aβ42/40 ratio; Aβ42 is more prone to oligomerization than Aβ40 and is the major component of plaques found in Alzheimer's diseased brains. However, an important note is that not all EOFAD PS1 mutations lead to increased production of AB42. Some mutations cause pick disease or FTD in absence of amyloid plaques. The G183V mutation causes Pick's disease in the absence of amyloid plaques, while other mutations cause frontotemporal dementia with varying degrees of AD-like neuropathology. Thus, the expression of AD or FTD phenotypes may depend on the degree of PS1 loss-of-function.

The majority of efforts aimed at treating Alzheimer's Disease (AD) have focused on reducing the symptoms of AD. In particular, identification of a lower concentration of choline acetyltransferase in affected neurons of the forebrains of AD patients has lead to treatments aimed at inhibiting the hydrolysis of acetylcholine in the synaptic cleft and prolonging the level of acetylcholine at the synapse. Although this strategy has resulted in at least a partial correction of neurotransmitter levels, the therapeutic benefits have been small.

Further, AD is categorized as a tauopathy. Tauopathies are caused by abnormal hyperphosphorylation of tau promoting its aggregation and formation of neurofibrillary tangles (NFTs). Since mutations in tau and APP both cause dementia, one or both may contribute to the disease progression of AD. It is well understood that mutations leading to altered processing of APP cause AD. Currently, there are no approved therapies for slowing the progression of Alzheimer's disease. Thus, there remains a need for more beneficial AD treatments. While most therapies in development are focused on altering APP metabolism, such as by γ-secretase inhibition, the present invention provides a treatment that addresses presenilin loss-of-functions caused by mutations in PSEN1 and PSEN2 (Table 2) using pharmacological chaperones which bind and increase presenlin levels thereby increasing presenilin function, rather than inhibiting γ-secretase activity.

All citations herein are incorporated by reference in their entirety.

### SUMMARY OF THE INVENTION

The present invention relates to a method for treating Alzheimer's Disease, comprising administering to the individual an effective amount of a pharmacological chaperone, wherein the pharmacological chaperone binds presenilin and as a result increases presenilin function and/or γ-secretase activity.

The invention further relates to a method for the treatment of a condition resulting from the pathological aggregation of tau protein in an individual by administering to the individual an effective amount of a pharmacological chaperone wherein the pharmacological chaperone binds presenlin and thereby increases presenilin function and/or γ-secretase activity.

The present invention also relates to a method for the treatment of depression in an individual, comprising administering to the individual an effective amount of a pharmacological chaperone, wherein the pharmacological chaperone binds presenelin and thereby increases presenilin function an/or γ-secretase activity.

### BRIEF DESCRIPTION OF THE DRAWINGS

FIG. 1: represents a schematic for APP processing.

FIG. 2: represents screening gels of γ-secretase inhibitors for presenilin-targeted pharmacological chaperones.

FIG. 3: depicts a western blot showing a dose-response for mature PS1 levels in SH-SY5Y neuroblastoma cells treated with the presenilin-targeted pharmacological chaperones G-IX, G-XXI and G-XX.

FIG. 4: time course experiment showing precursor enhancement using the pharmacological chaperone G-X.

FIG. 5A: depicts a western blot showing effects of presenilin-targeted pharmacological chaperones on precursor and mature PS1 levels.

FIG. 5B: is a graph of Western Blot data from FIG. 5A.

FIG. 6: depicts a Western Blot showing a dose-response effect for the presenilin-targeted pharmacological chaperone G-IV on γ-secretase levels.

FIG. 7: is a graph showing PS1, APP and Aβ40 levels from fibroblasts treated with the presenilin-targeted pharmacological chaperone G-XXI.

FIG. 8: is a time course graph showing effects of the presenilin-targeted pharmacological chaperone compound A on PS1, α-CTFs, Aβ40 and Aβ42 levels in the brains of C57BL6 mice.

### DETAILED DESCRIPTION OF THE INVENTION

The invention is directed to a method for the prevention and/or treatment of Alzheimer's Disease in an individual at risk for developing or diagnosed with the same, by administering to the individual an effective amount of a pharmacological chaperone which binds presenilin and thereby increases presenilin function and/or γ-secretase activity. The present invention may also be used to treat an individual suffering from or predisposed to depression. In one embodiment presenilin is presenilin 1 (PS1). In another embodiment presenilin is presenilin 2 (PS2). In another embodiment presenilin is both presenilin 1 (PS1) and presenilin 2 (PS2). In a further embodiment, the individual with or at risk for developing Alzheimer's Disease has a mutation in one or both of PSEN1 or PSEN2. The mutation(s) in PSEN1 or PSEN2 may be a misense mutation. In another embodiment, the individual may be deficient in presenilin 1 presenilin 2 or both presenilin 1 and presenilin 2.

### DEFINITIONS

The terms used in this specification generally have their ordinary meanings in the art, within the context of this invention and in the specific context where each term is used. Certain terms are discussed below, or elsewhere in the specification, to provide additional guidance to the practitioner in describing the invention and how to make and use the invention.

As used herein, the term “pharmacological chaperone,” or sometimes “specific pharmacological chaperone” (“SPC”), refers to a molecule that specifically binds to presenilin, and has one or more of the following effects: (i) enhancing the formation of a stable molecular conformation of the protein; (ii) enhances proper trafficking of the protein from the ER to another cellular location, preferably a native cellular location, i.e., preventing ER-associated degradation of the protein; (iii) preventing aggregation of conformationally unstable, i.e., misfolded proteins; (iv) restoring or enhancing at least partial wild-type function, stability, and/or activity of the protein; and/or (v) improving the phenotype or function of the cell (vi) stabilizing holo-presenilin, (vii) enhancing the stability and function of the γ-secretase complex. Thus, a pharmacological chaperone for presenilin is a molecule that binds to presenilin, resulting in proper folding, trafficking, non-aggregation, and increased activity of γ-secretase. It includes specific binding molecules, e.g., active site-specific chaperones, inhibitors, modulators, allosteric binders, non-active site binders that enhance protein stability.

The term “Pharmacological chaperones” (used interchangeable with the abbreviation “PCs”) refers to small molecules that selectively bind and stabilize target proteins to facilitate proper folding, reduce premature degradation and increase the efficiency of ER export. The small molecules are called “chaperones” because they help the proteins get from the where they are synthesized (the ER) to their intended location, in this case, the Golgi, endosomes, lysosomes, plasma membrane or any other intracellular compartment where presenilin is found outside the ER. The molecules are reversible binders which bind and stabilize the protein target, help restore proper trafficking, and then dissociate so the protein can carry out its proper function. The “pharmacological” modifier denotes molecular specificity: the molecules are designed to interact with and stabilize only a single intended protein target, and PCs do not generally affect multiple proteins or cellular processes such as protein trafficking, ER quality control, proteasome function, or the activity of biological chaperones (such as the heat shock proteins). This approach is broadly applicable to diseases where increasing the function of a specific protein (mutant or wild-type) is predicted to provide therapeutic benefit.

The retention and premature degradation of incorrectly folded proteins is not restricted to mutant proteins. It has been shown that a large fraction (up to 30%) of all newly synthesized proteins is targeted for premature degradation by the proteasomes. Subsequent studies have shown that pharmacological chaperones can increase cellular levels for many wild-type proteins by promoting protein folding, stability and ER export.

The term Aβ is used herein to refer to a hydrophobic 38- to 43-amino acid peptide, which is derived from the progressive enzymatic cleavage of a larger type I membrane protein, the amyloid precursor protein (APP).

The term “gamma-secretase” and “γ-secretase” are used herein interchangeably and refer to a multiprotein enzymatic complex that is responsible for generating the different Aβ species from truncated APP called c-terminal fragments (CTFs), which is generated by either alpha or beta secretase activity.

The term “PSEN1” refers to a gene that encodes encode γ-secretase proteins presenilin 1. The term “presenilin1” and “PS1” are used herein interchangeably. The term “PSEN21” refers to a gene that encodes encode γ-secretase proteins presenilin 2. The term “presenilin 2” and “PS2” are used herein interchangeably.

As used herein, the term “specifically binds” refers to the interaction of a pharmacological chaperone with presenilin (either presenilin 1, 2, or both), specifically, an interaction with amino acid residues of presenilin that directly participate in contacting the pharmacological chaperone. A pharmacological chaperone specifically binds a target protein, such as presenilin, to exert a chaperone effect on the enzyme and not a generic group of related or unrelated proteins. The amino acid residues of presenilin that interact with any given pharmacological chaperone may or may not be within the protein's “active site.” It may, for instance be at the substrate binding site. Specific binding, although complicated to show, can be evaluated through routine binding assays (e.g. inhibition, thermal stability) or through structural studies, e.g., co-crystallization, NMR, and the like, however, such binding is not easily shown in the γ-secretase complex because there is no co-crystallization and thermostability will not give specificity within a complex. Photoactivity inhibitor derivatives can be used to show specificity, but with some difficulty.

As used herein, the terms “enhance stability” or “increase stability” refers to increasing an enzymes resistance to irreversible inactivation in vitro or in a cell contacted with a pharmacological chaperone specific for presenilin (preferably of the same cell-type or the same cell, e.g., at an earlier time), that are not contacted with the pharmacological chaperone. Increasing protein stability increases the half-life of the protein in the ER and the amount of functional protein trafficked from the ER. In one aspect of the invention the stability of wild type presenilin is enhanced or increased resulting in increased levels of presenilin and γ-secretase. In another aspect of the invention the conformational stability of mutant presenilin is enhanced or increased resulting in increased levels

As used herein, the terms “enhance trafficking” or “increase trafficking” refer to increasing the efficiency of the transport of presenilin from the ER to the Golgi, endosomes, lysosomes, plasma membrane or any other intracellular compartment where presenlin is found outside of the ER, of a cell contacted with a pharmacological chaperone specific to presenilin, relative to the efficiency of transport of presenilin in a cell (preferably of the same cell-type or the same cell, e.g., at an earlier time) not contacted with the pharmacological chaperone specific for presenilin.

As used herein, the terms “enhance activity”, “enhance function”, “increase activity”, “increase function” refer to increasing the activity of presenilin and/or γ-secretase, as described herein, in a cell contacted with a pharmacological chaperone specific for presenilin and/or γ-secretase, relative to the activity of presenilin and/or γ-secretase in a cell (preferably of the same cell-type or the same cell, e.g., at an earlier time) not contacted with the pharmacological chaperone specific for presenilin and/or γ-secretase. Pharmacological chaperones of the present invention may also increase or enhance activity by increasing the total amount of presenilin in the cell and/or by increasing presenilin's specific activity. Examples of presenilin activity or function are listed in, but not limited to those listed in, Table 1.

The term “specific activity” refers to the amount of substrate an enzyme converts per milligram of protein in an enzyme preparation, per unit of time.

As used herein, the terms “enhance level” or “increase level” refer to increasing the amount of precursor or mature presenilin in a cell contacted with a pharmacological chaperone specific for presenilin, relative to the amount of presenilin in a cell (preferably of the same cell-type or the same cell, e.g., at an earlier time) not contacted with the pharmacological chaperone specific for presenilin.

The term “stabilize a proper conformation” refers to the ability of a presenilin pharmacological chaperone to induce or stabilize a conformation of a mutated or wild type presenilin that is functionally identical to the conformation of the wild-type presenilin that performs its intended function.

The term “functionally identical” means that while there may be minor variations in the conformation (almost all proteins exhibit some conformational flexibility in their physiological state), conformational flexibility does not result in (1) protein aggregation, (2) elimination through the endoplasmic reticulum-associated degradation pathway, (3) impairment of protein function, e.g., APP metabolic activity, and/or (4) improper transport within the cell, e.g., localization to the cytosol, to a greater or lesser degree than that of the wild-type protein.

The term “stable molecular conformation” refers to a conformation of a protein, i.e., presenilin, induced by a pharmacological chaperone that provides at least partial wild-type function in the cell or to enhance wild-type function. For example, a stable molecular conformation of presenilin would be one where presenilin leaves the ER and traffics to the Golgi, endosomes, lysosomes, plasma membrane or any other intracellular compartment where presenlin is found outside of the ER, instead of misfolding and being degraded and/or not performing its intended function. In addition, a stable molecular conformation of a mutated presenilin may also possess full or partial activity. However, it is not necessary that the stable molecular conformation have all of the functional attributes of the wild-type protein.

The term “Dominant negative mutation” refers to a mutation whose gene product adversely affects the normal, wild-type gene product within the same cell. For example, because EOFAD caused by mutations in presenilin is always inherited in an autosomal dominant manner, wild-type and mutant forms of presenilin are expressed in the same cell and therefor compete for inclusion in the gamma-secretase complex.

The term “presenilin” refers to the proteins presenilin 1 and presenlin 2 as a component of or independent of the γ-secretase complex.

The term “γ-secreatase activity” refers to the proteolytic processing of all γ-secretase substrates (e.g., APP, Notch, ErbB4, LRP1).

The term “activity” refers to the normal intended physiological function of presenilin or γ-secretase in a cell. For example, presenilin is a multifunctional protein that's involved in a number of different cellular processes including nuclear signal transduction, protein trafficking, intracellular calcium homeostasis, hippocampal neurogenesis and apoptosis. Such functionality of presenilin can be tested by any means known in the art to establish functionality.

In one non-limiting embodiment, presenilin polypeptide may be encoded for by any nucleic acid molecule exhibiting 50%, 60%, 70%, 80% and up to 100% homology to the nucleic acid molecules encoding a wild type presenilin, and any sequences which hybridize under standard conditions to these sequences. In another non-limiting embodiment, any other nucleotide sequence that encodes presenilin (having the same functional properties and binding affinities as the aforementioned polypeptide sequences), such as allelic variants in normal individuals, that have the ability to achieve a functional conformation in the ER, achieve proper localization within the cell, and exhibit wild-type activity.

As used herein the term “mutant” presenilin refers to a presenilin polypeptide translated from a gene containing a genetic mutation that results in an altered presenilin amino acid sequence. In one embodiment, the mutation results in a presenilin protein that does not achieve a native conformation under the conditions normally present in the ER, when compared with wild-type presenilin, or exhibits decreased stability or activity as compared with presenilin. This type of mutation is referred to herein as a “conformational mutation,” and the protein bearing such a mutation is referred as a “conformational mutant.” The failure to achieve this conformation results presenilin protein being degraded or aggregated, rather than being transported through a normal pathway in the protein transport system to its native location in the cell or into the extracellular environment. In some embodiments, a mutation may occur in a non-coding part of the gene encoding presenilin that results in less efficient expression of the protein, e.g., a mutation that affects transcription efficiency, splicing efficiency, mRNA stability, and the like. By enhancing the level of expression of wild-type as well as conformational mutant variants of presenilin, administration of a presenilin pharmacological chaperone can ameliorate a deficit resulting from such inefficient protein expression.

Certain tests may evaluate attributes of a protein that may or may not correspond to its actual in vivo activity, but nevertheless are appropriate surrogates of protein functionality, and wild-type behavior in such tests demonstrates evidence to support the protein folding rescue or enhancement techniques of the invention. One such activity in accordance with the invention is appropriate transport of a functional presenilin from the endoplasmic reticulum (ER) to the Golgi, endosomes, lysosomes, plasma membrane or any other intracellular compartment where presenlin is found outside of the ER.

A “deficiency in presenilin” refers to an amount of presenilin that is less than the normal amount of presenilin.

The terms “endogenous expression” and “endogenously expressed” refers to the normal physiological expression of presenilin in cells in an individual not having or suspected of having a disease or disorder associated with presenilin deficiency, overexpression of a dominant negative mutant, or other defect, such as a mutation in presenilin nucleic acid or polypeptide sequence that alters, e.g., inhibits, its expression, activity, or stability. This term also refers to the expression of presenilin in cells or cell types in which it is normally expressed in healthy individuals, and does not include expression of a presenilin in cells or cell types, e.g., tumor cells, in which presenilin is not expressed in healthy individuals.

As used herein, the term “efficiency of transport” refers to the ability of a protein to be transported out of the endoplasmic reticulum to its native location within the cell, cell membrane, or into the extracellular environment.

A “competitive inhibitor” of an enzyme can refer to a compound which structurally resembles the chemical structure and molecular geometry of the enzyme substrate to bind the enzyme in approximately the same location as the substrate. Thus, the inhibitor competes for the same active site as the substrate molecule, thus increasing the Km. Competitive inhibition is usually reversible if sufficient substrate molecules are available to displace the inhibitor, i.e., competitive inhibitors can bind reversibly. Therefore, the amount of enzyme inhibition depends upon the inhibitor concentration, substrate concentration, and the relative affinities of the inhibitor and substrate for the active site.

The “precursor form of presenilin” or “holo form of presenilin” refers to the ˜57 kDa uncleaned ER form of presenilin as detected on a western blot. The “mature form of presenilin” refers to the post-ER cleaved form of the precursor presenilin. The mature form of presenlin is a heterodimer consisting of a ˜30 kDa NH2-terminal fragment (NTF) and a ˜20 kDa C-terminal fragment as detected on a western blot by the presence of the ˜30 kDa NTF. “High affinity” binding of a pharmacological chaperone to the precursor form of presenilin and “low affinity binding of a pharmacological chaperone to the mature form of presenilin refers to concentrations at which a specific pharmacological chaperone stabilizes and increases the levels of precursor presenilin without inhibiting γ-secretase activity.

Non-classical competitive inhibition occurs when the inhibitor binds remotely to the active site of an enzyme, creating a conformational change in the enzyme such that the substrate can no longer bind to it. In non-classical competitive inhibition, the binding of substrate at the active site prevents the binding of inhibitor at a separate site and vice versa. This includes allosteric inhibition.

A “linear mixed-type inhibitor” of an enzyme is a type of competitive inhibitor that allows the substrate to bind, but reduces its affinity, so the Km is increased and the Vmax is decreased.

A “non-competitive inhibitor” refers to a compound that forms strong bonds with an enzyme and may not be displaced by the addition of excess substrate, i.e., non-competitive inhibitors may be irreversible. A non-competitive inhibitor may bind at, near, or remote from the active site of an enzyme or protein, and in connection with enzymes, has no effect on the Km but decreases the Vmax. Uncompetitive inhibition refers to a situation in which inhibitor binds only to the enzyme-substrate (ES) complex. The enzyme becomes inactive when inhibitor binds. This differs from non-classical competitive inhibitors which can bind to the enzyme in the absence of substrate.

The term “Vmax” refers to the maximum initial velocity of an enzyme catalyzed reaction, i.e., at saturating substrate levels. The term “Km” is the substrate concentration required to achieve ½ Vmax.

An enzyme “enhancer” is a compound that binds to presenilin and increases the amount of substrate processed by increasing the amount of γ-secretase complex, by increasing the specific activity of the γ-secretase complex, or by restoring normal processing of γ-secretase substrates, e.g., site of proteolytic cleavage, normalization of the Aβ1-42/Aβ1-40 ratio.

The terms “therapeutically effective dose” and “effective amount” refer to an amount that results in an over all net gain in enzymatic/protein function. With some molecules it may be possible to use doses that stabilize the ER form with out inhibiting γ-secreatase activity. “Therapeutically effective dose” and “effective amount” can also refer to an amount sufficient to enhance protein processing in the ER (permitting a functional conformation), or without inducing ligand-mediated receptor internalization of protein from the appropriate cellular location (in the case of an agonist), and enhance activity of the target protein, thus resulting in a therapeutic response in a subject. A therapeutic response may be any response that a user (e.g., a clinician) will recognize as an effective response to the therapy, including the foregoing symptoms and surrogate clinical markers. Thus, a therapeutic response will generally be an amelioration or inhibition of one or more symptoms of a disease or disorder, e.g., Alzheimer's Disease.

The phrase “pharmaceutically acceptable” refers to molecular entities and compositions that are physiologically tolerable and do not typically produce untoward reactions when administered to a human. Preferably, as used herein, the term “pharmaceutically acceptable” means approved by a regulatory agency of the federal or a state government or listed in the U.S. Pharmacopeia or other generally recognized pharmacopeia for use in animals, and more particularly in humans. The term “carrier” refers to a diluent, adjuvant, excipient, or any vehicle with which the compound is administered. Such pharmaceutical carriers, for example, can be sterile liquids, such as water and oils. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin, 18th Edition, or other editions.

The terms “about” and “approximately” shall generally mean an acceptable degree of error for the quantity measured given the nature or precision of the measurements. Typical, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

As used herein, the term “isolated” means that the referenced material is removed from the environment in which it is normally found. Thus, an isolated biological material can be free of cellular components, i.e., components of the cells in which the material is found or produced. In the case of nucleic acid molecules, an isolated nucleic acid includes a PCR product, an mRNA band on a gel, a cDNA, or a restriction fragment. In another embodiment, an isolated nucleic acid is preferably excised from the chromosome in which it may be found, and more preferably is no longer joined to non-regulatory, non-coding regions, or to other genes, located upstream or downstream of the gene contained by the isolated nucleic acid molecule when found in the chromosome. In yet another embodiment, the isolated nucleic acid lacks one or more introns. Isolated nucleic acids include sequences inserted into plasmids, cosmids, artificial chromosomes, and the like. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated organelle, cell, or tissue is removed from the anatomical site in which it is found in an organism. An isolated material may be, but need not be, purified.

The term “purified” as used herein refers to material, such as a nucleic acid or polypeptide that has been isolated under conditions that reduce or eliminate unrelated materials, i.e., contaminants. For example, a purified protein is preferably substantially free of other proteins or nucleic acids with which it is associated in a cell. As used herein, the term “substantially free” is used operationally, in the context of analytical testing of the material. Preferably, purified material substantially free of contaminants is at least 50% pure; more preferably, at least 90% pure, and more preferably still at least 99%) pure. Purity can be evaluated by conventional means, e.g., chromatography, gel electrophoresis, immunoassay, composition analysis, biological assay, and other methods known in the art.

The term “Tauopathy” refers to any condition resulting from the pathological aggregation of tau protein forming neurofibrillary tangles (NFT) in the human brain and includes (but is not limited to) diseases such as Frontotemporal dementia, Alzheimer's disease, Progressive supranuclear palsy, Corticobasal degenerations and frontotemporal lobar degeneration (Pick's disease).

The term “Alzheimer's Disease” or “AD” refers to a condition characterized by slowly progressive dementia and gross cerebral cortical atrophy. The presence of β-amyloid neuritic plaques, intra neuronal neurofibrillary tangles, and amyloid angiopathy are hallmarks of AD and are observed at postmortem examination. AD may be heritable in a Familial manifestation, or may be sporadic. Herein, AD includes Familial, Sporadic, as well as intermediates and subgroups thereof based on phenotypic manifestations. Familial AD typically has an early-onset (before age 65) while Sporadic AD typically is late-onset (age 65 and later). In a non-limiting embodiment, Familial AD may be associated with mutations in one or more genes selected from the group comprising presenilin 1 (human presenilin 1, GenBank Accession Nos. NM_{—}000021, NM_{—}007318, and NM_{—}007319; murine presenilin 1, GenBank Accession No. NM_{—}008943; and rat presenilin 1, GenBank Accession No. NM_{—}019163), presenilin 2 (human presenilin 2, GenBank Accession Nos. NM_{—}000447, and NM_{—}012486; murine presenilin 2, GenBank Accession No. NM_{—}011183; and rat presenilin 2, GenBank Accession No. NM_{—}031087), and Amyloid Precursor Protein (APP) (human APP, GenBank Accession Nos. NM_{—}201414, NM_{—}201413, and NM_{—}000484; murine APP, GenBank Accession No. NM_{—}007471; and rat APP, GenBank Accession No. NM_{—}019288). Sporadic AD can not be tested for directly, but certain risk factors may increase an individual's susceptibility to developing sporadic AD. In one, non-limiting embodiment, individuals with at least one copy of the e4 allele of Apolipoprotein E (APOE) (human APOE, GenBank Accession No. NM_{—}000041; murine APOE, GenBank Accession No. NM_{—}009696; and rat APOE, GenBank Accession No. NM_{—}138828) are at risk of developing late-onset sporadic AD.

This term also includes individuals with trisomy 21, or Down syndrome (DS), develop dementia that is identical to the clinical and neurophathogic characteristics of AD (in their third or fourth decade), including cerebral amyloid (Aβ) plaques and neurofibrillary tangles (NFTs), the characteristic lesions of Alzheimer disease (AD). Recent studies have shown that the Aβ42 is the earliest form of this protein deposited in Down syndrome brains, and may be seen in subjects as young as 12 years of age, and that soluble Aβ can be detected in the brains of DS subjects as early as 21 gestational weeks of age, well preceding the formation of Aβ plaques. Gyure et al., Archives of Pathology and Laboratory Medicine 125: 489-492 (2000).

This term further includes individuals with depression. Studies have linked neurogenesis to the beneficial actions of specific antidepressants, suggesting a connection between decreased hippocampal neurogenesis and depression. (Malberg et al. 2000. J Neurosci. 20 (24): 9104-10; Manev H. et al. 2001. Eur J Pharmacol. 411 (1-2): 67-70)

For purposes of the present invention, a “neurological disorder” refers to any central nervous system (CNS) or peripheral nervous system (PNS) disease that is associated with the processing of a gamma-secretase substrate or a loss of presenilin function. This may result in neuronal or glial cell defects including but not limited to neuronal loss, neuronal degeneration, neuronal demyelination, gliosis (i.e., astrogliosis), neuronal or extraneuronal accumulation of aberrant proteins or toxins (e.g., amyloid-β), reduced or impaired neurogenesis.

According to the present invention, one exemplary neurological disorder is cerebral amyloid angiopathy (CAA), also referred to as amyloid angiopathy. This disorder is a form of angiopathy in which the same amyloid protein that is associated with Alzheimer's disease, amyloid-β (Aβ), deposits in the walls of the leptomeninges and superficial cerebral cortical blood vessels of the brain. Amyloid deposition predisposes these blood vessels to failure, increasing the risk of a hemorrhagic stroke. Since it is the same amyloid protein that is associated with Alzheimer's dementia, such brain hemorrhages are more common in people who suffer from Alzheimer's, however they can also occur in those who have no history of dementia. The hemorrhage within the brain is usually confined to a particular lobe and this is slightly different compared to brain hemorrhages which occur as a consequence of high blood pressure (hypertension)—a more common cause of a hemorrhagic stroke (or cerebral hemorrhage). CAA is also associated with transient ischemic attacks, subarachnoid hemorrhage, Down syndrome, post irradiation necrosis, multiple sclerosis, leucoencephalopathy, spongiform encephalopathy, and dementia pugilistica.

The term “individual” “patient” or “patient population” refers to a person(s) diagnosed as having Alzheimer's Disease or at risk of developing Alzheimer's Disease. For instance, the individuals are diagnosed, or at risk of developing Familial AD. In another instance, the individual is diagnosed as having, or at risk of developing, Sporadic AD. Diagnosis of AD may be made based on genotypic or phenotypic characteristics displayed by the individual. For example, an individual with a mutant variant of presenilin 1, presenilin 2, or APP are at risk of developing familial AD. In another, non-limiting example, individuals with the E4 variant of APOE are at risk for developing Sporadic AD.

According to the present invention, an individual may be diagnosed as having AD, or at risk of developing AD, by having a mutation in genes APP, PSEN1 or PSEN2 or by exhibiting phenotypes associated with AD. Phenotypes associated with AD may be cognitive or psychiatric. Examples of cognitive phenotypes include, but are not limited to, amnesia, aphasia, apraxia and agnosia. Examples of psychiatric symptoms include, but are not limited to, personality changes, depression, hallucinations and delusions. As one non-limiting example, the Diagnostic and Statistical Manual of Mental disorders, 4th Edition (DSM-IV-TR) (published by the American Psychiatric Association) contains the following set of criteria for dementia of the Alzheimer's type:
A. The development of multiple cognitive deficits manifested by both memory impairment and one or more of Aphasia, Apraxia, Agnosia and disturbances in executive functioning;
B. The cognitive deficits represent as decline from previous functioning and cause significant impairment in social or occupational functioning;
C. The course is characterized by gradual onset and continuing decline;
D. The cognitive deficits are not due to other central nervous system, systemic, or substance-induced conditions that cause progressive deficits in memory and cognition; and
E. The disturbance is not better accounted for by another psychiatric disorder.

Another non-limiting example is The National Institute of Neurological and Communicative Disorders and Stroke-Alzheimer's Disease and Related Disorder Association (NINDS-ADRDA) Criteria for Alzheimer's Disease as follows:
A. Definite Alzheimer's disease: meets the criteria for probable Alzheimer's disease and has histopathologic evidence of Alzheimer's disease via autopsy or biopsy
B. Probable Alzheimer's disease: dementia established by clinical and neuropyschological examination and involves
(a) progressive deficits in two or more areas of cognition, including memory,
(b) onset between the ages of 40 and 90 years, and
(c) absence of systemic or other brain diseases capable of producing a dementia syndrome, including delirium
C. Possible Alzheimer's disease: a dementia syndrome with an atypical onset, presentation, or progression and without a known etiology; any co-morbid diseases capable of producing dementia are not believed to be the cause
D. Unlikely Alzheimer's disease: a dementia syndrome with any of the following: sudden onset, focal neurologic signs, or seizures or gait disturbance early in the course of the illness.

Phenotypic manifestations of AD may also be physical, such as by the direct (imaging) or indirect (biochemical) detection of amyloid-β plaques. Quantitation of amyloid-β (1-40) in the peripheral blood has been demonstrated using high-performance liquid chromatography coupled with tandem mass spectrometry in a linear ion trap (Du et al., J Biomol Tech. 16(4):356-63 (2005). Detection of single β-amyloid protein aggregates in the cerebrospinal fluid of Alzheimer's patients by fluorescence correlation spectroscopy also has been described (Pitschke et al., Nature Medicine 4: 832-834 (1998). U.S. Pat. No. 5,593,846 describes a method for detecting soluble amyloid-β. Indirect detection of amyloid-β peptide and receptor for advanced glycation end products (RAGE) using antibodies also has been described. Lastly, biochemical detection of increased BACE-1 activity in cerebrospinal fluid using chromogenic substrates also has been postulated as a diagnostic or prognostic indicator of AD (Verheijen et al., Clin Chem. Apr 13 [Epub.] (2006).

In vivo imaging of β-amyloid can be achieved using radioiodinated flavone derivatives as imaging agents, Ono et al., J Med Chem. 48(23):7253-60 (2005), and with amyloid binding dyes such as putrescein conjugated to a 40-residue radioiodinated A peptide (yielding ¹²⁵I-PUT-A 1-40), which was shown to cross the blood-brain barrier and bind to αβ plaques. Wengenack et al., Nature Biotechnology. 18(8):868-72 (2000). Imaging of β-amyloid was also shown using stilbene [¹¹C]SB-13 and the benzothiazole [¹¹C]6-OH-BTA-1 (also known as [¹¹C]PIB). Nicholaas et al., Am J Geriatr Psychiatry, 12:584-595 (2004).

The present invention is directed to treating an individual who may be diagnosed with Alzheimer's Disease or may be at risk for developing Alzheimer's Disease. In one aspect of the invention, Alzheimer's Disease is early onset familial Alzheimer's Disease. The present invention may be used to treat any form of Alzheimer's Disease at any stage of the disease.

According to the present invention, the pharmacological chaperone can bind the precursor form of presenilin with high or low affinity and the mature form of presenilin with high or low affinity. In one embodiment the pharmacological chaperone binds the precursor form of presenilin with high affinity and the mature form of presenilin with low affinity. Or, the pharmacological chaperone can bind one form of presenilin and not the other form. The pharmacological chaperone may stabilize the precursor form of presenilin and the pharmacological chaperone may increase the function of presenilin.

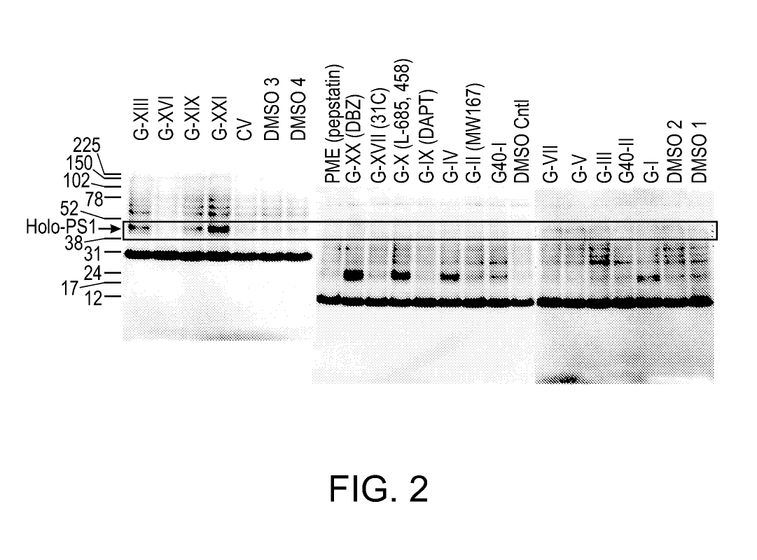

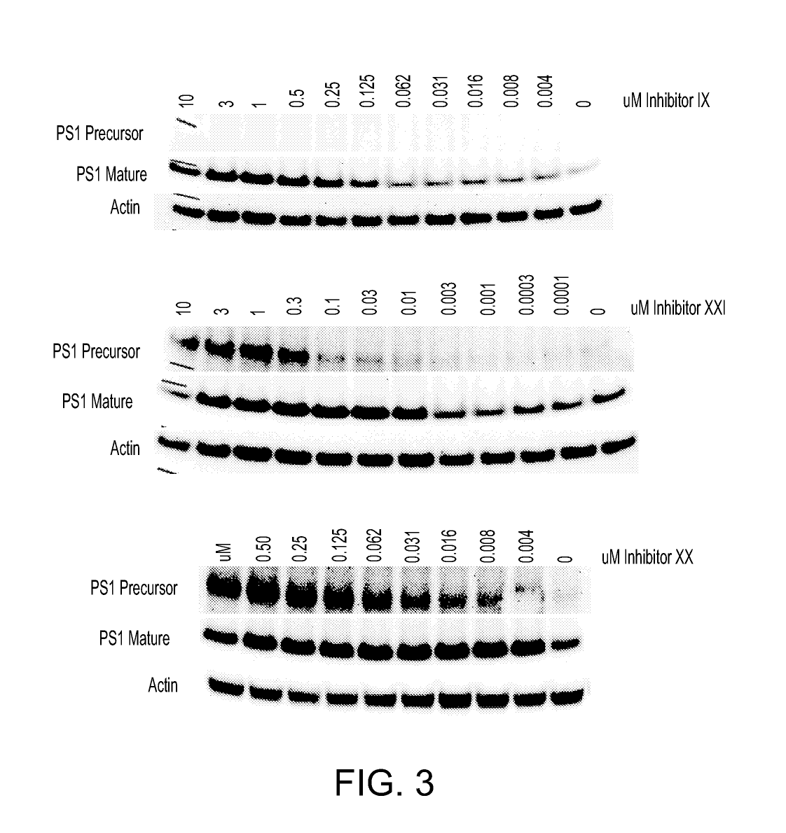

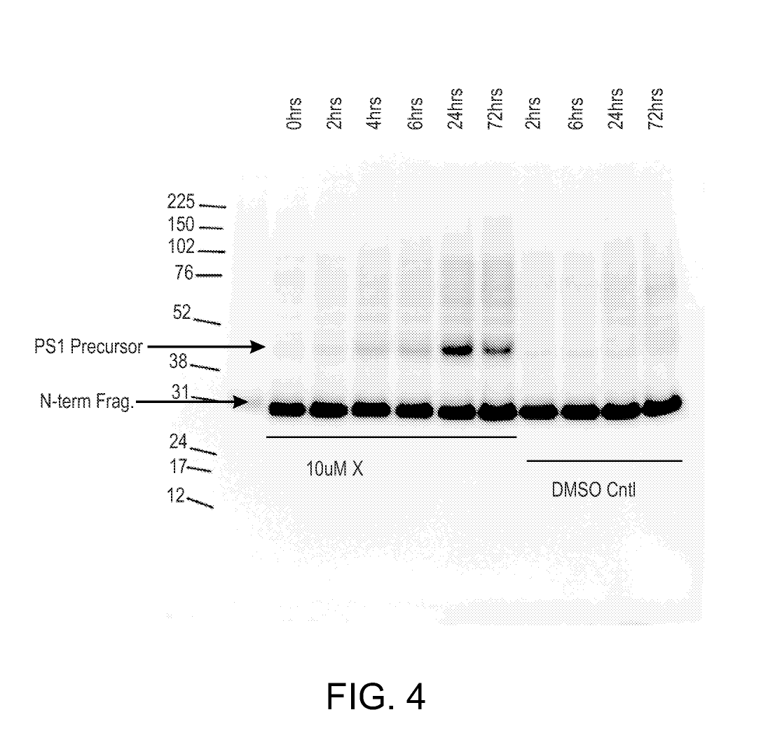

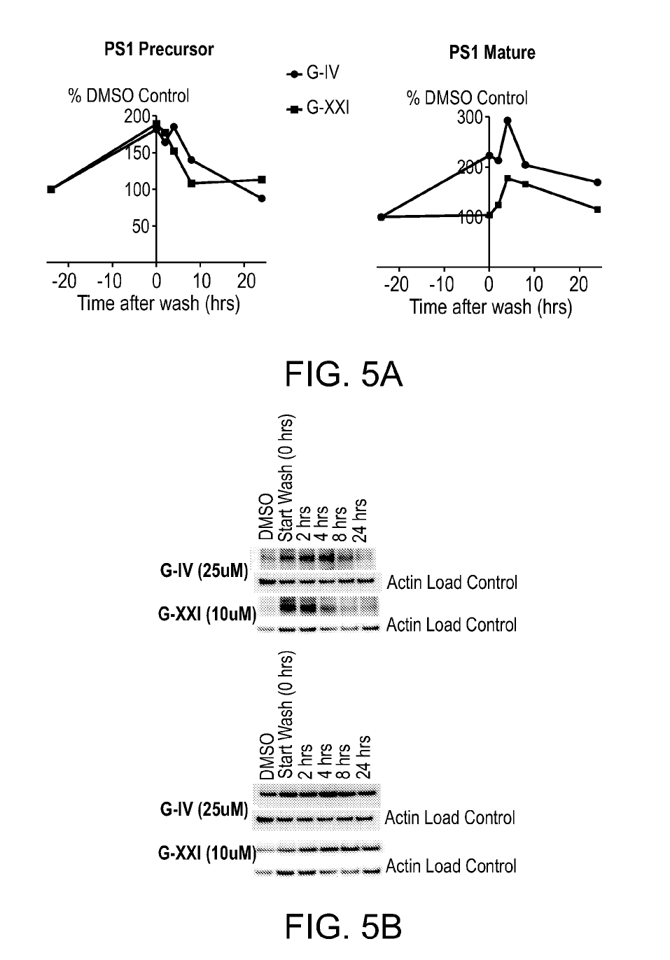

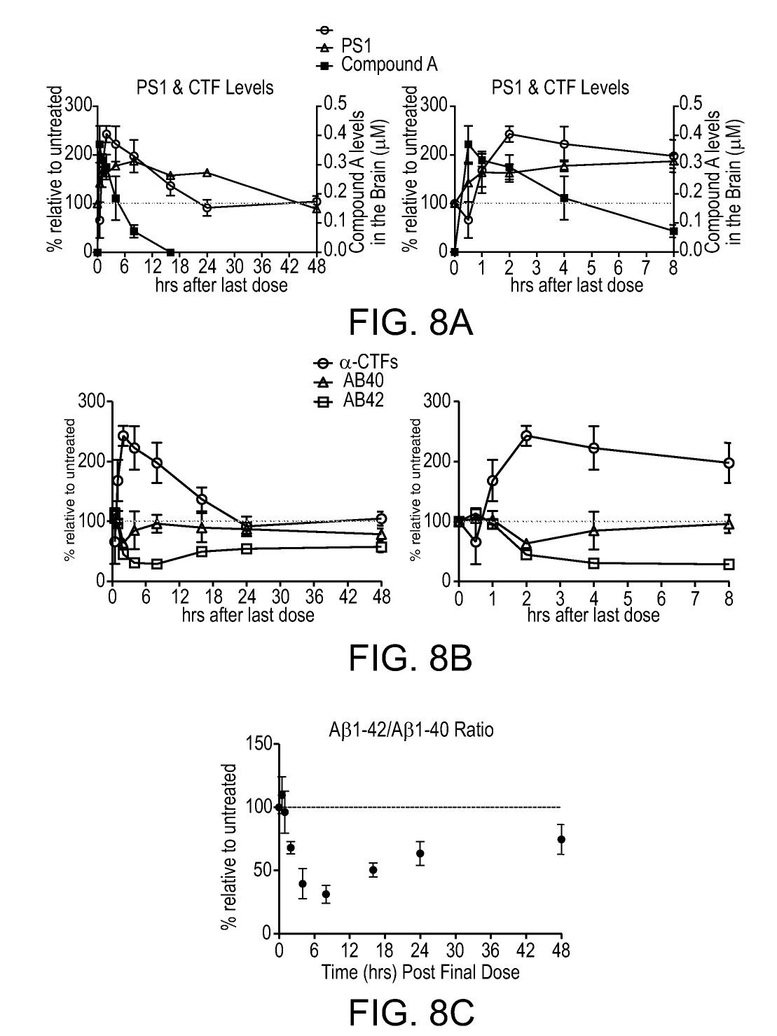

Pharmacological chaperones (PCs) are small molecules that selectively bind and stabilize target proteins to facilitate proper folding, reduce premature degradation and increase the efficiency of ER export. Select gamma-secretase inhibitors can stabilize the ER precursor form of presnilin (FIGS. 2 and 3; Table 3). The presenilin precursor has a short half life in the ER, so when it is stabilized via pharmacological chaperones, levels increase rapidly (FIG. 4). Additionally, precursor presenilin can be rapidly converted into mature presenilin in the presence of the PC or once the PC has been washed out (FIG. 5), demonstrating that the gamma-secretase inhibitors are functioning as a pharmacological chaperone for presenilin. The rapid increase in precursor presenilin levels and conversion to mature presenlin, combined with the relatively long half-life of mature presenilin (t_{1/2}˜24 hrs), allows for a net increase in presenelin activity if PCs with γ-secretase inhibitory activity are dose appropriately. As a result, for compounds with a relatively short half-life in the brain (≦8 hours), dosing for a short period of time may be best, such as one day on and 1-2 days off. Using a γ-secretase inhibitor as a pharmacological chaperone through stabilizing the presenilin precursor would most likely have a short 1-2 day treatment (increasing presenilin precursor) followed by 1-3 days of non-treatment to allow for precursor to mature presenilin conversion and increased activity of γ-secretase complex. This intermittent dosing strategy would not be expected to effectively inhibit gamma-secretase activity therefore limiting its potential therapeutic benefit in the context of an amyloid reduction therapy. Furthermore, intermittent dosing may reduce side effects associated with inhibiting presenilin activity (e.g., notch inhibition). This type of dosing strategy is supported by our in vivo study where mice were treated once daily for 4 days with a PC (compound A) that has a relatively short half-life (brain t_{1/2}˜3.5 hrs) followed by a 2 day washout period (FIG. 8). During the washout period, presenilin levels rapidly increased by 50% within 1 hour and maximum increase of 2-fold with in 8 hours. Brain levels of compound A reached subinhibitory levels (IC₅₀˜60 nM) post final dose, while presenilin levels remained at 50% above untreated levels at 24 hours resulting in a net gain in presenilin activity. Declining CTF levels confirm minimal inhibition of γ-activity activity after 2 hrs into the washout. Aβ42 levels remain 40% reduced relative to untreated controls 48 hrs after the final dose. In the absence of inhibitory concentrations of compound A, it would expected that Aβ42 levels would return to untreated control levels. The prolonged reduction of Aβ42 and the reduced Aβ42/Aβ40 ratio indicates this effect is a result of the PC effect on presenilin. In the case of using a γ-secretase inhibitor for a slow increase in mature presenilin, a dosing regimen more typical of enhancing lysosomal enzymes would be more appropriate.

In one embodiment, the pharmacological chaperone can increase mutant and wild type presenilin. Some mutations may reduce presenilin function through a dominant negative mechanism. In another embodiment, the pharmacological chaperone can increase wild type presenilin levels without increasing the levels of mutant presenilin.

In one aspect of the invention, the increase in presenilin function leads to an increase in the trafficking of APP to the plasma membrane. Further, the invention is also directed to a method for the treatment of a condition resulting from the pathological aggregation of tau protein in an individual via the administering to the individual an effective amount of a pharmacological chaperone, wherein the pharmacological chaperone binds presenlin and thereby increases γ-secretase activity. Such a condition may be, for example, Frontotemporal dementia, Alzheimer's disease, Progressive supranuclear palsy, corticobasal degenerations and frontotemporal lobar degeneration, or other similar or related disorders or diseases.

According to the present invention, individuals to be treated may have any form of Alzheimer's Disease, including early onset familial Alzheimer' Disease. The individual may have a risk factor for Alzheimer's Disease or be at risk for developing Alzheimer's Disease. The individual may have already been diagnosed with Alzheimer's Disease. Further, the individual may not have been diagnosed, but displays hallmarks of the disease. The individual may have or may be at risk for developing Progressive supranuclear palsy, Corticobasal degenerations and/or Frontotemporal lobar degeneration (Pick's disease). In a further embodiment, Alzheimer's Disease is caused by or linked to Down syndrome or another related disorder.

Further, the claimed treatment may include combinations of one or more pharmacological chaperones and may also be combined with other known Alzheimer treatments or any other treatments used to treat conditions related to or resulting from the pathological aggregation of tau protein. PS1 is required for neurogenesis and the lack of neurogenesis may lead to depression and/or the early onset of Alzheimer's Disease. The claimed treatment may also be used in combination with other pharmaceuticals or treatments known to promote neurorgenesis to treat depression and Alzheimer's Disease. The claimed invention may be combined with enzyme replacement therapies, substrate deprevation therapies or any combination therefore.

The present invention is also directed to a method for the treatment of a condition resulting from the pathological aggregation of Tau protein, such as Frontotemporal demential, Progressive supranuclear palsy, Corticobasal degenerations and Frontotemporal lobar degeneration or other related or similar diseases or disorders.

In the present invention, pharmacological chaperones will be dosed to maximize presenilin levels and to minimize inhibition of γ-secretase. γ-secretase inhibitors will be dosed less frequently to establish a pharmacological chaperone effect (e.g., increase presenilin 1 and/or 2 levels and function) compared to when the same inhibitors are used with the therapeutic intention of inhibiting γ-secretase activity. Therefore the pharmacological chaperone will be dosed one time per week, twice per week, three times per week, four days per week, five days per week, or even less or more frequently as needed.

In one aspect of the invention, Compound A will be dosed once every other day, every third day, every forth day, every fifth day or once weekly. In another aspect of the invention, compound A will be dosed for 1 to 4 consecutive days, followed by 1 to 5 days without drug.

Many mutant forms of presenilin have a lower affinity for γ-secretase inhibitors compared to wild type presenilin. In another aspect of the invention, a pharmacological chaperone will be dosed to specifically increase wild-type presenilin without increasing mutant presenilin levels to avoid the potential dominant negative effect of presenilin mutants. One example of a pharmacological chaperone useful in the present invention is ((2S)-2-hydroxy-3-methyl-N-((1S)-1-methyl-2-{[(1S)-3-methyl-2-oxo-2,3,4,5-tetrahydro-1H-3-benzazepin-1-yl]amino}-2-oxoethyl)butanamide), which is commercially available from Acesys Pharma Tech Limited, located in North Brunswick, N.J. Further examples of pharmacological chaperones that may be useful in the present invention can be found in Table 2:

TABLE 2
Pharmacological chaperones for presenilin

| Compound | |
|---|---|
| (with IUPAC name | |
| as generated by | |
| ChemBioDraw Ultra v. | |
| 12.0 and other known | |
| common names) | Chemical Structure |
| Compound A ((S)-1- (((S)-5-methyl-6-oxo- 6,7-dihydro-5H- dibenzo[b,d]azepin-7- yl)amino)-1-oxopropan- 2-yl (2,2,3,3,3- pentafluoropropyl)carbamate) | 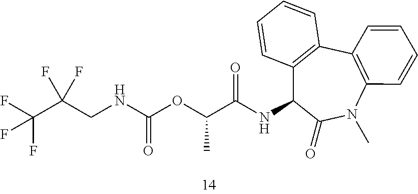 [SMILES: C[C@H](OC(=O)NCC(F)(F)C(F)(F)F)C(=O)N[C@@H]1C(=O)N(C)c2ccccc2-c2ccccc21] |
| Compound B ((R)-4-(2- (1-(4-chloro-N-(2,5- difluorophenyl)phenyl- sulfonamido)ethyl)-5- fluorophenyl)butanoic acid) | 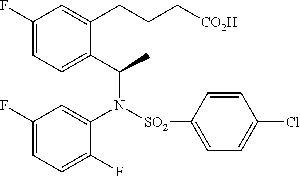 |
| G-I (I) (benzyl ((2R)-4- methyl-1-(((2S)-4- methyl-1-oxo-1-((1- oxohexan-2- yl)amino)pentan-2- yl)amino)-1-oxopentan- 2-yl)carbamate) | 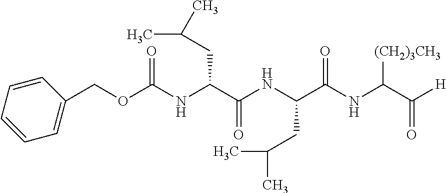 |
| G-II (II) ((11S)-19-tert- butyl 1-methyl 2,14-di- sec-butyl-9,9-difluoro- 5,17-diisopropyl-11- methyl-4,8,10,15,18- pentaoxo-3,6,7,12,13,16- hexaazanon) | 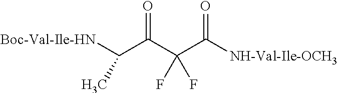 |
| G-IV (IV) (N-((S)-3- methyl-1-oxo-1-(((S)-1- oxo-3-phenylpropan-2- yl)amino)butan-2-yl)-2- naphthamide) | 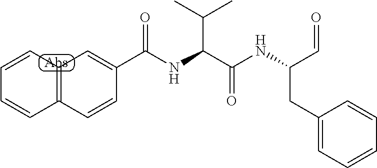 [SMILES: CC(C)[C@H](NC(=O)c1ccc2ccccc2c1)C(=O)N[C@H](C=O)Cc1ccccc1] |
| G-IX; DAPT (IX) ((S)- tert-butyl 2-((S)-2-(2-(3,5- difluorophenyl)acetamido) propanamido)-2- phenylacetate) | 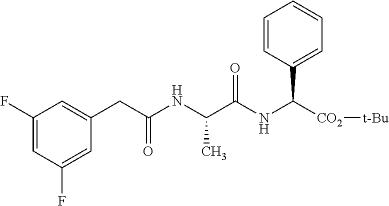 |
| G-X (X) (tert-butyl ((2S,3R,5R)-6-(((S)-1- (((S)-1-amino-1-oxo-3- phenylpropan-2- yl)amino)-4-methyl-1- oxopentan-2-yl)amino)- 5-benzyl-3-hydroxy-6- oxo-1-phenylhexan-2- yl)carbamate) | 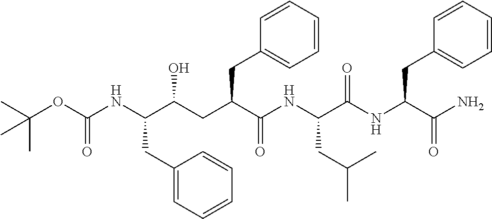 [SMILES: CC(C)C[C@H](NC(=O)[C@H](Cc1ccccc1)C[C@@H](O)[C@H](Cc1ccccc1)NC(=O)OC(C)(C)C)C(=O)N[C@@H](Cc1ccccc1)C(N)=O] |
| G-XIII (XIII) (benzyl ((2S)-3-(4- hydroxyphenyl)-1-((3- methyl-1-(((S)-4-methyl- 1-oxopentan-2- yl)amino)-1-oxopentan- 2-yl)amino)-1- oxopropan-2- yl)carbamate) | 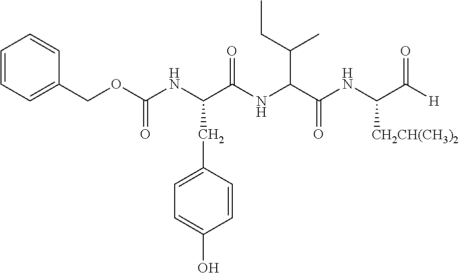 |
| G-XVII (XVII) ((6S,7R,12S,15S)-methyl 6,9-dibenzyl-7-hydroxy- 12-isobutyl-15- isopropyl-2,2-dimethyl- 4,10,13-trioxo-3-oxa- 5,9,11,14- tetraazahexadecan-16- oate) | 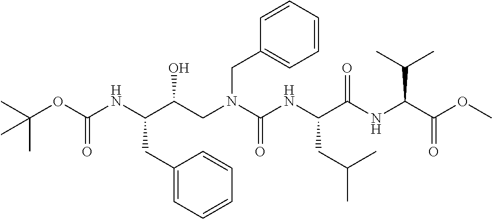 [SMILES: COC(=O)[C@@H](NC(=O)[C@H](CC(C)C)NC(=O)N(Cc1ccccc1)C[C@@H](O)[C@H](Cc1ccccc1)NC(=O)OC(C)(C)C)C(C)C] |
| G-XIX (XIX) ((2R,3R)- 3-(3,4-difluorophenyl)- 2-(4-fluorophenyl)-4- hydroxy-N-((S)-2-oxo-5- phenyl-2,3-dihydro-1H- benzo[e][1,4]diazepin-3- yl)butanamide) | 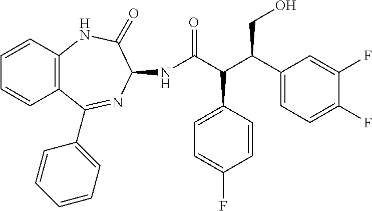 |
| G-XX; DBZ (XX) ((S)- 2-(2-(3,5-difluoro- phenyl)acetamido)- N-((S)-5-methyl-6- oxo-6,7-dihydro-5H- dibenzo[b,d]azepin-7- yl)propanamide) | 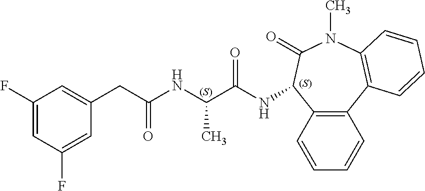 |
| G-XXI; Compound E (XX1) ((2S)-2-(2-(3,5- difluorophenyl)acetamido)- N-((3R)-1-methyl-2- oxo-5-phenyl-2,3,4,5- tetrahydro-1H- benzo[e][1,4]diazepin-3- yl)propanamide | 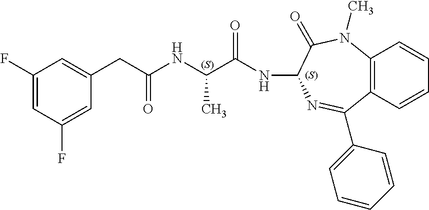 |
| Compound C: ((S)-2- hydroxy-3-methyl-N- ((S)-1-(((S)-3-methyl-2- oxo-2,3,4,5-tetrahydro- 1H-benzo[d]azepin-1- yl)amino)-1-oxopropan- 2-yl)butanamide) | 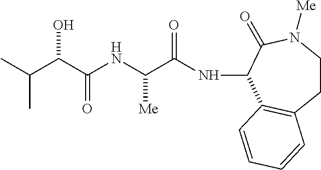 [SMILES: CC(C)[C@H](O)C(=O)N[C@@H](C)C(=O)N[C@@H]1C(=O)N(C)CCc2ccccc21] |
| Compound H ((2S)-2- hydroxy-3-methyl-N- ((1S)-1-methyl-2-{[(1S)- 3-methyl-2-oxo-2,3,4,5- tetrahydro-1H-3- benzazepin-1-yl]amino}- 2-oxoethyl)butanamide) | 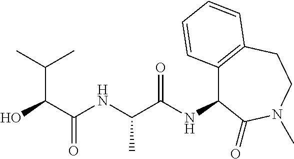 [SMILES: CC(C)[C@H](O)C(=O)N[C@@H](C)C(=O)N[C@@H]1C(=O)N(C)CCc2ccccc21] |

Presenilin 1 and 2 are highly homologous (74%) polytopic membrane proteins consisting of nine transmembrane domains and possesses the active site for γ-secretase proteolytic activity. Due to this homology, a pharmacological chaperone which binds presenilin 1 will most likely bind presenilin 2.

Molecular Chaperones Stabilize Protein Conformation.

In the human body, proteins are involved in almost every aspect of cellular function. Certain human diseases result from mutations that cause changes in the amino acid sequence of a protein which reduce its stability and may prevent it from folding properly. The majority of genetic mutations that lead to the production of less stable or misfolded proteins are called missense mutations. These mutations result in the substitution of a single amino acid for another in the protein. Because of this error, missense mutations often result in proteins that have a reduced level of biological activity. In addition to missense mutations, there are also other types of mutations that can result in proteins with reduced biological activity.

Proteins generally fold in a specific region of the cell known as the endoplasmic reticulum, or ER. The cell has quality control mechanisms that ensure that proteins are folded into their correct three-dimensional shape before they can move from the ER to the appropriate destination in the cell, a process generally referred to as protein trafficking. Misfolded and/or unstable proteins are often eliminated by the quality control mechanisms after initially being retained in the ER. In certain instances, misfolded proteins can accumulate in the ER before being eliminated.

The retention of misfolded proteins in the ER interrupts their proper trafficking, and the resulting reduced biological activity can lead to impaired cellular function and ultimately to disease. In addition, the accumulation of misfolded proteins in the ER may lead to various types of stress on cells, which may also contribute to cellular dysfunction and disease.

Endogenous molecular chaperones are present in virtually all types of cells and in most cellular compartments. Some are involved in the transport of proteins and permit cells to survive under stresses such as heat shock and glucose starvation. Among the endogenous chaperones (molecular chaperones), BiP (immunoglobulin heavy-chain binding protein, Grp78) is the best characterized chaperone of the ER. Like other chaperones, BiP interacts with many secretory and membrane proteins within the ER throughout their maturation. When nascent protein folding proceeds smoothly, this interaction is normally weak and short-lived. Once the native protein conformation is achieved, the molecular chaperone no longer interacts with the protein. BiP binding to a protein that fails to fold, assemble, or be properly glycosylated becomes stable, and usually leads to degradation of the protein through the ER-associated degradation pathway. This process serves as a “quality control” system in the ER, ensuring that only those properly folded and assembled proteins are transported out of the ER for further maturation, and improperly folded proteins, or unstable proteins, are retained for subsequent degradation. Due to the combined actions of the inefficiency of the thermodynamic protein folding process and the ER quality control system, only a fraction of some wild-type proteins become folded into a stable conformation and successfully exit the ER.

Pharmacological Chaperones Derived from Specific Enzyme Inhibitors Rescue Mutant Enzymes and Enhance Wild-Type Enzymes.

The binding of small molecule inhibitors of enzymes associated with lysosomal storage diseases (LSDs), for instance, can increase the stability of both mutant enzyme and the corresponding wild-type enzyme (see U.S. Pat. Nos. 6,274,597; 6,583,158; 6,589,964; 6,599,919; 6,916,829, and 7,141,582 all incorporated herein by reference). In particular, it was discovered that administration of small molecule derivatives of glucose and galactose, which are specific, selective competitive inhibitors for several target lysosomal enzymes, effectively increased the stability of the enzymes in cells in vitro and, thus, increased trafficking of the enzymes to the lysosome. Thus, by increasing the amount of enzyme in the lysosome, hydrolysis of the enzyme substrates is expected to increase. Since the mutant enzyme protein is unstable in the ER, the enzyme protein is retarded in the normal transport pathway (ER→4 Golgi apparatus→endosomes→lysosome) and prematurely degraded. Therefore, certain compounds which binds to and increases the stability of a mutant enzyme, may serve as “chaperones” for the enzyme and increase the amount that can exit the ER and move to the lysosomes.

Since some enzyme inhibitors are known to bind specifically to the catalytic center of the enzyme (the “active site”), resulting in stabilization of enzyme conformation in vitro, these inhibitors were proposed, somewhat paradoxically, to be effective chaperones that could help restore exit from the ER, trafficking to the lysosomes, hydrolytic activity. These specific pharmacological chaperones were designated “active site-specific chaperones (ASSCs)” or “specific pharmacological chaperones” since they bound in the active site of the enzyme in a specific fashion. Pharmacological chaperone therapy has potential advantages over enzyme replacement therapy (ERT) since a small molecule can be orally administered and may have superior biodistribution compared to protein-based therapies.

In addition to rescuing the mutant enzymes, the pharmacological chaperones enhance ER secretion and activity of wild-type enzymes. Thus, a compound that induces a stable molecular conformation of an enzyme during folding serves as a “chaperone” to stabilize the enzyme in a proper conformation for exit from the ER.

In the present application, binding of a pharmacological chaperone to ER (precursor) form of presenilin can restore or increase γ-secretase enzymatic activity and as a result can increase trafficking of full length APP to the plasma membrane surface for increased alpha processing by alpha-secretase and less beta processing by BACE, increasing sAPPalpha (soluble amyloid precursor protein alpha) and decreasing Aβ; decreasing the Aβ1-42/Aβ1-40 ratio, restore autophagic and macroautophagic function; restore calcium homeostasis; increase neurogenesis; increase neurite outgrowth and/or increase synaptogenesis; decrease neuronal apoptosis; decrease tau hyperphosphorylation and/or neurofibrillary tangles. It has been shown that if a mutation occurs in presenilin, then more Aβ is formed in the transgolgi network and thereby less sAPPα is generated at the cell surface. Binding of a pharmacological chaperone to presenilin and thereby restoring γ-secretase enzymatic activity may increase the processing of other γ-secretase substrates. γ-secretase has many substrates, potentially as many as 100 or more. PS1 knockout mice leads to a reduction of neurogenesis, a way of generating new neurons, and an increase in neurogenesis has been demonstrated in mice when PS1 increases.

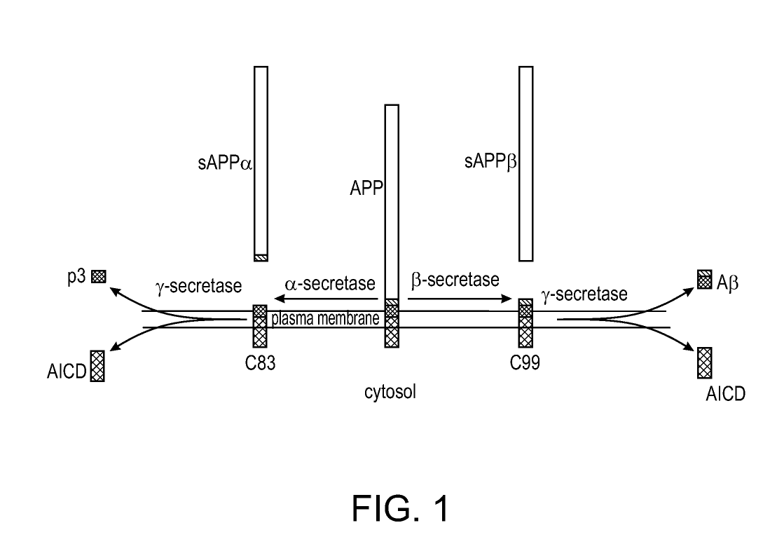

FIG. 1: shows the processing of APP. As shown in this figure, mature APP is metabolized by two competing pathways, the α-secretase pathway that generates sAPPa and C83 (left) and the β-secretase pathway that generates sAPPβ and C99 (right). Carboxyterminal fragments C83 and C99 are substrates for γ-secretase, generating the APP intracellular domain (AICD) and the secreted peptides p3 (left) and Aβ (right). Aβ peptides can oligomerize, form plaques and promote tau hyperphosphorylation, while sAPPα promotes neurite outgrowth, synaptogenesis and suppresses tau hyperphosphorylation associated with CDK5 activation.
FIG. 2: shows the results of screening different γ-secretase inhibitors for PS1 Holo (precursor) enhancement and SH-SY5Y neuroblastoma cells incubated with various γ-secretase inhibitors for 24 hrs.

### Methods for Generating Data in FIG. 2:

Eighteen different γ-secretase inhibitors (purchased from Calbiochem) were tested for their ability to stabilize the PS1 precursor. SH-SY5Y neuroblastoma cells (1e6) incubated in 6-well plates were dosed with ˜50 uM γ-secretase inhibitor (10% FBS, DMEM) for 24 hrs. Cells were lysed in 2% CHAPS (TBS+protease inhibitors), cell debris pelleted at 18,000 g. Total protein in cell lysates were determined by BCA and 25 ug of protein/lane loaded onto 4-12% BisTris NuPage gels (Invitrogen) that were electrophoresed at 150V for 1 hr. Gels were transferred to PVDF (25V, 1 hr) and probed with anti-N-term PS1 (1:1000, Abcam cat. #ab10281). Western blots were developed using chemiluminescence and imaged using an Alpha Innotech FluorChem Q imaging system. Γ-secretase inhibitors tested: PME, XX, XVII, X, IX, IV, II, I-40, VII, V, III, II-40, I, XIII, XVI, XIX, XXI, CW.

The mature form of PS1 (where PS1 is processed to form a heterodimer) is evidenced by a prominent band at 27 kDa (developed using an antibody specific for the N-terminus of PS1) and is present in all samples. The unprocessed or precursor form of PS1 is evidenced by a band at 45 kDa and is significantly increased when SY5Y neuroblastomas are treated with certain γ-secretase inhibitors (XX, X, IV, II, I-40, I, XIII, XIX and XXI). The increase in PS1 precursor protein level is interpreted as stabilization of the PS1 precursor and the stabilization appears to be mechanistically distinct from the inhibition of γ-secretase activity since there is no correlation between γ-secretase inhibitory potency (IC50 for Abeta production) and levels of stabilized PS1 precursor (see Table 1). This result is evidence that the binding site at which the PS1 precursor is stabilized is pharmacologically distinct from the “active site” of mature PS1 in an active γ-secretase complex.

TABLE 3
Comparison of IC50 values of γ-secretase inhibitors tested and their ability to stabilize the PS1 precursor
(IC50 in uM; NA = not available).

| | | PS1 | |
|---|---|---|---|
| | IC50 | Precursor | |
| γ-secretase | (Abeta | Enhance- | |
| Inhibitor | production) | ment | |
| PME | 0.150 | − | 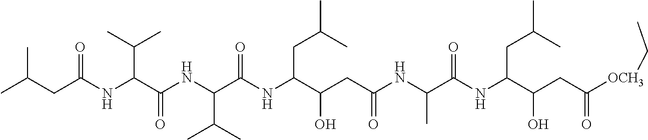 |
| XX (G-XX) | 0.0017 | +++ | See Table 2 |
| XVII | 0.300 | − | See Table 2 |
| (G-XVII) | | | |
| X (G-X) | 0.017 | +++ | See Table 2 |
| IX (G-IX) | 0.115 | − | See Table 2 |
| IV (G-IV) | 2.6 | ++ | See Table 2 |
| II (G-II) | 13 | + | See Table 2 |
| I-40 | 15 | − | trans-3,5-DMC-Ile-Leu-CHO |
| | | | (DMC = Dimethoxycinnamoyl) |
| VII (G-VII) | 2.3 | − | 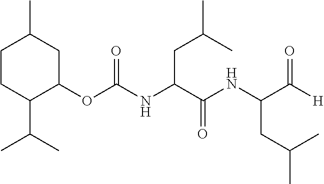 [SMILES: CC(C)CC(C=O)NC(=O)C(CC(C)C)NC(=O)OC1CC(C)CCC1C(C)C] |
| V (G-V) | 5 | − | 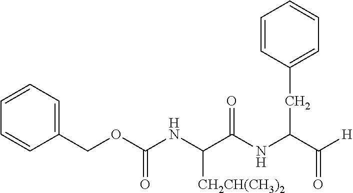 |
| III (G-III) | 35 | − | 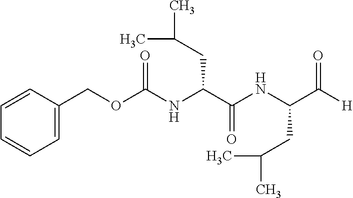 [SMILES: CC(C)C[C@@H](C=O)NC(=O)[C@@H](CC(C)C)NC(=O)OCc1ccccc1] |
| II-40 | NA | − | 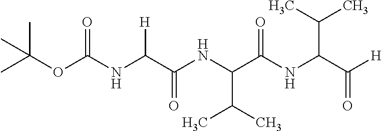 [SMILES: CC(C)C(C=O)NC(=O)C(NC(=O)CNC(=O)OC(C)(C)C)C(C)C] |
| I (G-I) | NA | + | See Table 2 |
| XIII | 0.008 | + | See Table 2 |
| (G-XIII) | | | |
| XVI (G-XVI) | 0.010 | − | 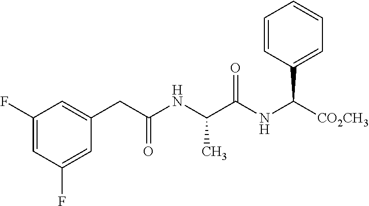 [SMILES: COC(=O)[C@@H](NC(=O)[C@H](C)NC(=O)Cc1cc(F)cc(F)c1)c1ccccc1] |
| XIX (G-XIX) | 0.00006 | + | 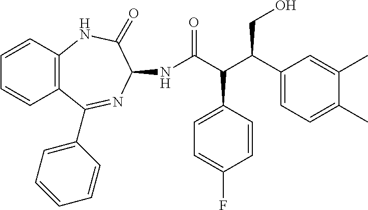 |
| XXI | 0.0003 | ++ | See Table 2 |
| (G-XXI) | | | |
| CW | NA | − | 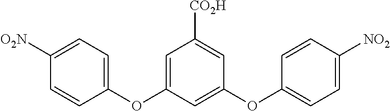 [SMILES: O=C(O)c1cc(Oc2ccc([N+](=O)[O-])cc2)cc(Oc2ccc([N+](=O)[O-])cc2)c1] |

PS1 Precursor Enhancement is classified as follows:
− no effect
+ some effect
++ moderate effect
+++ strong effect

FIG. 3. Mature PS1 dose response to a four day treatment with γ-secretase inhibitors IX, XXI and XX on holo and mature PS1 levels in cells dosed for four days.

### Methods for Generating Data in FIG. 3:

SY5Y neuroblastomas grown in 6-well plates were dosed with different concentrations of γ-secretase inhibitors IX, XXI, and XX for four days, after which the cells were harvested by centrifugation (5000 g, 5 min.), lysed in RIPA buffer (TBS, 1% TX100, 0.5% taurodeoxycholate, 0.1% SDS plus protease inhibitors) and cleared of unsolubilized cell debris by centrifugation (15,000 g, 10 min., 4 C). Total protein was determined by BCA and equivalent protein loads of 50 ug/lane were separated on a 4-12% BisTris NuPage polyacrylamide gel (Invitrogen) then transferred to PVDF (25V, 1 hr in NuPage Transfer buffer). Western blots were probed with anti-N-term PS1 (1:1000, Abcam cat. #ab10281) followed by development using an anti-rabbit alkaline phosphatase conjugate, chemiluminescence detection and imaged using an Alpha Innotech FluorChem Q imaging system. After PS1 bands were quantified using the FluorChem Q band analysis software, blots were stripped and re-probed for actin (rabbit anti-actin, Pierce Chemicals).

Western blot results show that SY5Y neuroblastomas exhibit a γ-secretase inhibitor dose dependent increase in mature PS1 protein levels after four days of treatment. These results also show that the slower increase in mature PS1 levels do not appear to be dependent on PS1 precursor stabilization since γ-secretase inhibitor IX, which does not stabilize the PS1 precursor, shows similar dose response behavior as the γ-secretase inhibitors XX and XXI, which do stabilize the PS1 precursor. Also, the effective doses of the inhibitors are closer to the known IC50s (Abeta production) for the inhibitor. Therefore, it seems that the inhibitor is most likely having its effect on mature PS1 levels through the interaction with PS1 in the mature γ-secretase complex and the mechanism of action is different than stabilization of the PS1 precursor. This also means that dosing can be done at lower concentrations (compared to PS1 precursor stabilization using the same inhibitors) and perhaps chronic dosing at sub-IC50 levels of inhibitor.

FIG. 4: Time course of PS1 precursor enhancement using 10 uM γ-secretase inhibitor for EOFAD patient fibroblasts treated with the γ-secretase inhibitor G-X (10 uM) over a 72 hour time period.

### Methods for Generating Data in FIG. 4:

Lymphoblast cell line AG07622 was dosed in 6-well plates with 10 uM of γ-secretase inhibitor X (in 10% FBS, DMEM) for 0 hrs, 2 hrs, 4 hrs, 6 hrs and 24 hrs. Control samples containing 1% DMSO were ran in parallel to the drug treated lymphoblasts. After the specified dosing time, cells were harvested by centrifugation (5000 g, 5 min.), lysed in 2% CHAPS/TBS (with protease inhibitors) and cleared of unsolubilized cell debris by centrifugation (15,000 g, 10 min., 4 C). Total protein was determined by BCA and equivalent proteins loads of 40 ug/lane were separated on a 4-12% BisTris NuPage polyacrylamide gel (Invitrogen) then transferred to PVDF (25V, 1 hr in NuPage Transfer buffer). Western blots were probed with anti-N-term. PS1 (1:1000, Abcam cat. #ab 10281) followed by development using an anti-rabbit alkaline phosphatase conjugate, chemiluminescence detection and imaged using an Alpha Innotech FluorChem Q imaging system.

As early as 2 hrs, an increase in PS1 precursor is evident as shown in an increase in intensity of the 45 kDa band. The PS1 precursor level continues to increase up to a maximum that is reached after 24 hrs. The fact that PS1 enhancement is seen over short time periods (hours versus days) indicates that the PS1 precursor is being stabilized since it is known to have a much shorter half-life than the mature PS1 found in the γ-secretase complex.

FIG. 5: Presenilin pharmacological chaperone gamma-secretase inhibitor wash-out experiment. A. Quantification of western blot data for PS1 precursor and PS1 mature over a period of 24 hours of washing. B. Western blot data of PS1 precusor, PS1 mature form and corresponding actin load controls used for quantification in A.

### Method for Generating Data in FIG. 5:

SH-SY5Y neuroblastoma cells were treated for 24 hours with various presenilin pharmacological chaperones, then the drug was removed and levels of PS1 precursor and PS1 mature were measured at 0, 2, 4, 8 and 24 hours after removal of the drug.

SY5Y neuroblastomas in 6-well plates were treated with γ-secretase inhibitors IV (25 uM), X (1 uM), XXI (10 uM), II (14 uM), XX (10 uM) and a DMSO control for 24 hrs. After the 24 hr treatment, neuroblastomas were washed every 2 hrs (up to 8 hrs and then a 24 hr time point) by a full media exchange (2 ml 10% FBS in DMEM). At each time point cells were harvested by centrifugation (5000 g, 5 min.), lysed in 2% CHAPS/TBS (with protease inhibitors) and cleared of unsolubilized cell debris by centrifugation (15,000 g, 10 min., 4 C). Total protein was determined by BCA and equivalent proteins loads of 40 ug/lane were separated on a 4-12% BisTris NuPage polyacrylamide gel (Invitrogen) then transferred to PVDF (25V, 1 hr in NuPage Transfer buffer). Western blots were probed with anti-N-term PS1 (1:1000, Abeam cat. #ab10281) followed by development using an anti-rabbit alkaline phosphatase conjugate, chemiluminescence detection and imaged using an Alpha Innotech FluorChem Q imaging system. After PS1 bands were quantified using the FluorChem Q band analysis software, blots were stripped and re-probed for actin (rabbit anti-actin, Pierce Chemicals). PS1 precursor and mature PS1 (N-terminal fragment) were normalized against actin and subsequently reported as percent DMSO control prior to dosing.

In general, when SY5Y neuroblastomas are dosed for 24 hrs with the above inhibitors, the protein levels of the PS1 precursor increase by 50-100%. After the 24 hr dosing period when washing out of the γ-secretase inhibitor is initiated the PS1 precursor levels begin to fall towards baseline levels. However, with the falling PS1 precursor levels a concomitant increase of the mature form of PS1 is realized, upwards of 200% of basal levels. This result is evidence that the inhibitor stabilized PS1 precursor can be converted to the mature form of PS1 (found only in the active γ-secretase complex) when the drug is washed out, generating increased levels of γ-secretase. This is an example of how to increase γ-secretase levels by a pharmacological chaperone that binds and stabilizes the PS1 precursor. The stabilization of the PS1 precursor is pharmacologically distinct from inhibiting γ-secretase (see Data above and Table 1), one can find (screen existing γ-secretase inhibitor libraries etc.) a small molecule that binds and stabilizes the PS1 precursor with greater potency than inhibition of γ-secretase. Such a molecule would be an ideal PC for increasing levels of γ-secretase with minimal side effects due to inhibition of γ-secretase activity.

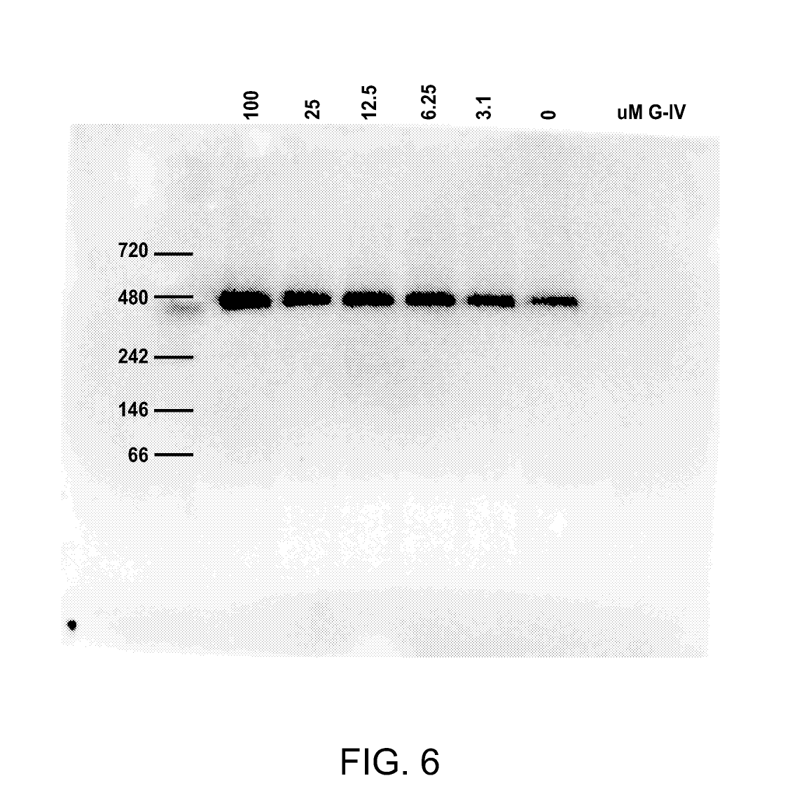

FIG. 6: Native gel of intact γ-secretase complex from fibroblasts treated with different doses of the presenilin pharmacological chaperone γ-secretase inhibitor IV. Increased presenilin levels (not shown) correlate with increased levels of the γ-secretase complex indicating that the chaperoned presenilin is getting incorporated into the mature γ-secretase complex.

### Methods for Generating Data in FIG. 6:

Wildtype fibroblasts (CRL2076) in a 6-well plate were treated with different doses of γ-secretase inhibitor IV (0, 3.1, 6.25, 12.5, 25 and 100 uM) for 24 hrs, followed by a 6 hr wash out period. Fibroblasts were harvested by gentle scraping in TBS, collected by centrifugation (5000 g, 5 min) and lysed in 1% digitonin (50 mM NaCl, 50 mM Hepes, pH 7.3) overnight at 4° C. Nonsolubilized cell debris was removed by centrifugation (15,000 g, 10 min.). Total protein was determined by BCA and equivalent proteins loads of 40 ug/lane were separated on a 4-16% native BisTris polyacrylamide gel using Blue-Native cathode and anode buffer system (Invitrogen). The native gel was transferred to PVDF (25V, 1 hr in NuPage Transfer buffer), blocked with Blotto (Pierce) and probed with anti-N-term PS1 (1:1000, Abcam cat. #ab10281) followed by development using an anti-rabbit alkaline phosphatase conjugate, chemiluminescence detection and imaged using an Alpha Innotech FluorChem Q imaging system.

PS1 specific immunoblotting of γ-secretase inhibitor treated fibroblast lysates separated under native conditions shows a single band near the 480 kDa molecular weight marker, very close to the expected molecular weight of 440 kDa for the intact γ-secretase complex (consisting of aph1, nicastrin, pen2 and PS1). The band intensity of the γ-secretase complex increases as a function of increasing concentration of the γ-secretase inhibitor. This dose response demonstrates that the increases in the mature form (N-terminal fragment) observed previously corresponds to an increase in γ-secretase complex; it also shows that a γ-secretase inhibitor can be used as a pharmacological chaperone to increase the level of γ-secretase complex.

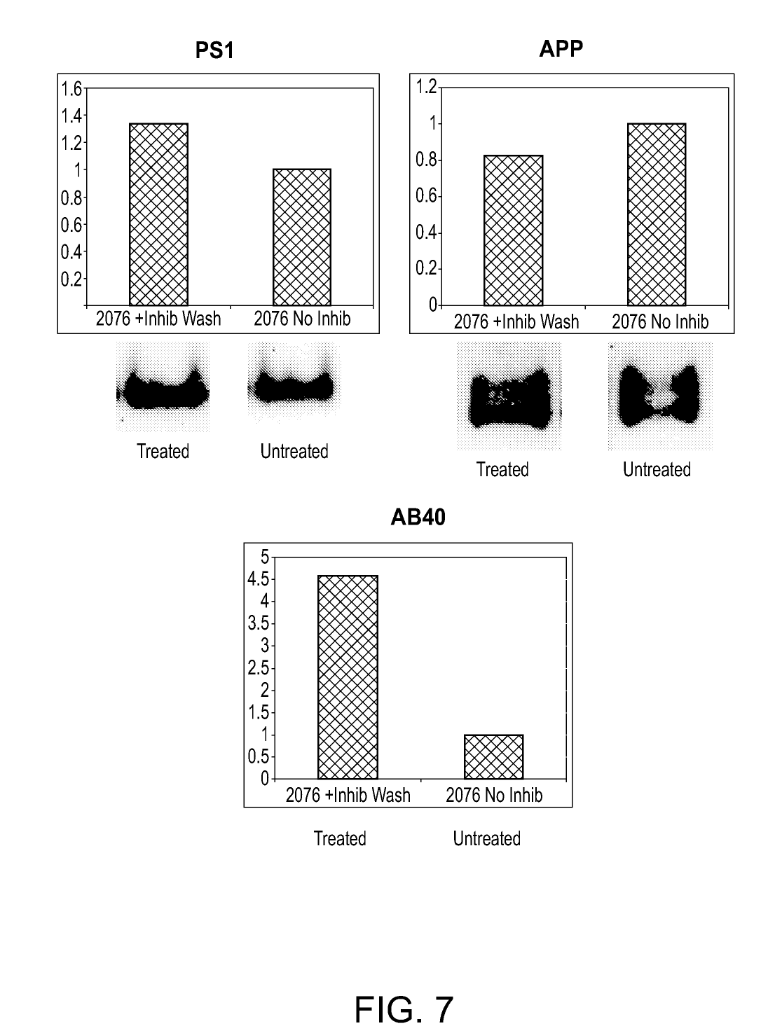

FIG. 7: Effect of PS1 enhancement increases endogenous APP substrate turnover (Abeta production). Fibroblasts derived from healthy humans were treated with the presenilin-targeted pharmacological chaperone G-XXI for 24 hours, drug was removed and the cells were cultured for another 24 hours. APP and Aβ (in the medium) levels were measured 24 hours after the drug was removed.

### Methods for Generating Data in FIG. 7:

Fibroblasts (AG06848) were grown to confluency in T-75 flasks where they were treated with γ-secretase inhibitor XXI (10 uM in 10% FBS/DMEM or no inhibitor for untreated sample) for 24 hrs. Cells were subsequently washed (complete media exchange) with repeated washes after 2 hrs and 4 hrs. Following the final wash, cells were incubated in 10 ml of serum-free DMEM for 20 hrs. Supernatants were removed, filtered through a 0.22 uM syringe filter and concentrated to 100 ul by centrifugation (4000 g, 90 min at 4 C) using a 3 kDa MWCO ultrafilter (Amicon). Abeta 40 levels were quantified from concentrated supernatants using an ABeta40 specific ELISA (Covance). Fibroblasts were lysed in RIPA buffer (TBS, 1% TX100, 0.5% taurodeoxycholate, 0.1% SDS plus protease inhibitors) and cleared of unsolubilized cell debris by centrifugation (15,000 g, 10 min., 4 C). Total protein was determined by BCA and equivalent protein loads of 50 ug/lane were separated on a 4-12% BisTris NuPage polyacrylamide gel (Invitrogen) then transferred to PVDF (25V, 1 hr in NuPage Transfer buffer). Western blots were probed with anti-N-term PS1 (1:1000, Abcam cat. #ab 10281); full length APP was detected using an antibody specific for the C-terminus of APP (Anaspec). Western blots were developed using an anti-rabbit alkaline phosphatase conjugate, chemiluminescence detection and imaged using an Alpha Innotech FluorChem Q imaging system. After PS1 bands were quantified using the FluorChem Q band analysis software, blots were stripped and re-probed for tubulin (1:1000 rabbit anti-tubulin, Abcam). Blots for PS1 and APP were normalized against tubulin levels and Abeta 40 ELISA results were normalized against total protein of fibroblast lysates from which the supernatants were derived.

When fibroblasts were dosed with γ-secretase inhibitor XXI for 24 hrs and followed by a 24 hr wash out period, enhanced levels of mature PS1 were still measured as well as a significant increase in the amount of AB40 secreted into the media. The increased production of Abeta indicates that the increased mature PS1 level translates into greater γ-secretase activity on one of its endogenous substrates, the c-terminal fragment (CTF) of APP. This demonstrates that γ-secretase activity can be increased through a pharmacological chaperone approach.

FIG. 8. In vivo enhancement of wild-type presenilin 1 in the brains of mice treated with the pharmacological chaperone compound A. In vivo effects of compound A on brain levels of PS1, Aβ, sAPPα, α-CTFs and full length APP. FIG. 8A is a time course showing brain levels of α-CTFs, PS1 and compound A up to 48 hours after the final dose of compound A was administered. FIG. 8B is a time course showing brain levels of α-CTFs, Aβ40 and Aβ42 up to 48 hours after the final dose of compound A was administered. FIG. 8C is a time course showing Aβ1-42/Aβ1-40 ratios in the brain up to 48 hours after the final dose of compound A was administered. CTF levels begin to drop after 2 hours indicating that gamma-secreatase is no longer inhibited, while brain levels of mature PS1 remain elevated for well after compound A and α-CTFs have returned to baseline levels.

### Methods for Generating Data in FIG. 8.

Mice were treated via gavage once daily for 3 consecutive days with 30 mg/Kg of compound A and then necropsied 0-48 hours after the final dose was administered.

C57BL6 mice were treated by oral gavage of 30 mg/kg compound A (0.5% methylcellulose carrier) once a day for 4 consecutive days. After the final dose, nine groups of animals (5 animals per group) were sacrificed at different time points ranging from 15 min. post final dose up to 48 hours post final dose (plus an untreated group). Blood was collected and mouse brain samples were minced and homogenized in water using a FastPrep homogenizer (Thermo Scientific). Plasma and brain homogenates were extracted with acetonitrile for measurement of compound A concentrations by LCMS. For biochemical analysis, brains were homogenized (250 mM sucrose, 50 mM Tris, pH 7.5, 1 mM EDTA plus protease inhibitors) and separated into a membrane fraction and soluble fraction by ultracentrifugation at 100,000 g for 1 hr. The membrane fraction was solubilized in RIPA buffer (1% TX100, 0.5% Taurodexoycholate, 0.1% SDS in TBS+PI) and clarified by centrifugation. Total protein was measured by BCA (Pierce) and 30 ug of each sample was separated by SDS/PAGE and analyzed by Western blot. The following antibodies were used: PS1 ( 1/1000 Abcam no. Ab10281); calnexin ( 1/1000 Abcam no. Ab10286) and APP-CTF ( 1/1000 Anaspec 54096). Western blots were developed using an alkaline phosphatase anti-rabbit secondary and an alkaline phosphatase chemiluminescent substrate. Chemiluminescence data was imaged using an Alpha Innotech FluorChem Q imager and band quantification was carried out using densitometry software AlphaViewQ (Alpha Innotech). For AB40 and AB42 measurements, brain homogenates were re-homogenized with an equal volume of 0.4% diethylamine/100 mM NaCl, clarified by ultracentrifugation at 100,000 g for 1 hr and neutralized using 1/10 volume of 0.5M Tris, pH 7. Abeta40 was quantified from diethylamine extracts using BetaMark Abeta40 ELISA kit (Covance); Abeta42 was quantified from diethylamine extracts using the High Sensitivity Abeta42 ELISA kit (Wako).

1. A method for treating Alzheimer's Disease in an individual, comprising administering to the individual an effective amount of a pharmacological chaperone, wherein the pharmacological chaperone binds presenilin and thereby increases one or more of presenilin function and γ-secretase activity.

2. (canceled)

3. The method of claim 1, wherein presenilin is presenilin-1.

4. The method of claim 1, wherein presenilin is presenilin-2.

5. The method of claim 1, wherein the pharmacological chaperone hinds the precursor form of presenilin with high affinity and the mature form of presenilin with low affinity.

6. The method of claim 1, wherein the individual has a mutation in the PSEN1 and/or PSEN2 gene.

7. The method of claim 1, wherein the individual is deficient in presenilin.

8. The method of claim 1, wherein the pharmacological chaperone increases mutant or wild type presenilin.

9. The method of claim 1, wherein Alzheimer's Disease is early onset familial Alzheimer's Disease.

10. The method of claim 1, wherein the increase in γ-secretase activity leads to an increase in the trafficking of amyloid precursor protein to the plasma membrane.

11. The method of claim 1, wherein the pharmacological chaperone is a compound selected from Table 2.

12. The method of claim 1, herein the pharmacological chaperone is G-XX.

13. A method for the treatment of a condition resulting from the pathological aggregation of tau protein in an individual, comprising administering to the individual an effective amount of a pharmacological chaperone, wherein the pharmacological chaperone binds presenilin and thereby increases one or more of presenilin function and γ-secretase activity.

14. (canceled)

15. The method of claim 13, wherein said condition is selected from the group consisting of Fronto temporal Dementia, Alzheimer's Disease, Progressive Supranuclear Palsy, Corticobasal Degenerations and Frontotemporal Lobar Degeneration.

16. A method for the treatment of depression in an individual, comprising administering to the individual an effective amount of a pharmacological chaperone, wherein the pharmacological chaperone binds presenilin and thereby increases one or more of presenilin function and γ-secretase activity.

17. (canceled)

18. The method of claim 11, wherein the pharmacological chaperone is compound A.
